(19) United States
(12) Patent Application Publication
(10) Pub. No.: US 2007/0117860 A1
(43) Pub. Date: May 24, 2007

(54) Bicyclic indolinesulphonamide derivatives

(75) Inventors: Elke Dittrich-Wengenroth, Wuppertal (DE); Hilmar Bischoff, Wuppertal (DE); Stephan Siegel, Wuppertal (DE); Michael Woltering, Hilden (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/566,340

(22) PCT Filed: Jul. 20, 2004
(86) PCT No.: PCT/EP04/08075

§ 371 Date: Dec. 18, 2006

(30) Foreign Application Priority Data
Aug. 2, 2003 (DE) 10335449.2

(51) Int. Cl. A61K 31/404; C07D 209/04

(52) U.S. Cl. 514/418; 548/465; 548/509

(57) ABSTRACT

The invention relates to novel bicyclic indoline sulfonamide derivatives of general formula (I), methods for the production thereof, and the use thereof in medicaments, especially as potent PPAR delta agonists for preventing and/or treating cardiovascular diseases, particularly dyslipidemia, arteriosclerosis, and coronary heart diseases.

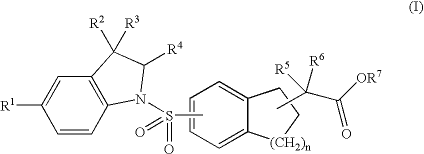

The present application relates to novel bicyclic indolinesulphonamide derivatives, to processes for their preparation and to their use in medicaments, in particular as potent PPAR-delta-activating compounds for the prophylaxis and/or treatment of cardiovascular disorders, in particular dyslipidaemias, arteriosclerosis and coronary heart diseases.

In spite of many successful therapies, coronary heart diseases (CHDs) remain a serious public health problem. Treatment with statins, which inhibit HMG-CoA reductase, very successfully lowers the LDL cholesterol plasma concentration, resulting in a significant reduction of the mortality of patients at risk; however, convincing treatment strategies for the therapy of patients having an unfavourable HDL/LDL cholesterol ratio and/or hypertriglyceridaemia are still not available to date.

Currently, fibrates are the only therapy option for patients of these risk groups. They act as weak agonists of the peroxisome-proliferator-activated receptor (PPAR)-alpha (Nature 1990, 347, 645-50). A disadvantage of fibrates which have hitherto been approved is that their interaction with the receptor is only weak, requiring high daily doses and causing considerable side-effects.

For the peroxisome-proliferator-activated receptor (PPAR)-delta (Mol. Endocrinol. 1992, 6, 1634-41), first pharmacological findings in animal models indicate that potent PPAR-delta-agonists may likewise lead to an improvement in the HDL/LDL cholesterol ratio and in hypertriglyceridaemia.

WO 00/23407 discloses PPAR modulators for treating obesity, atherosclerosis and/or diabetes. WO 93/15051 and EP 636 608-A1 describe 1-benzenesulphonyl-1,3-dihydroindol-2-one derivatives as vasopressin and/or oxytocin antagonists for the treatment of various disorders.

It was an object of the present invention to provide novel compounds suitable for use as PPAR-delta modulators.

The present invention provides compounds of the general formula (I)

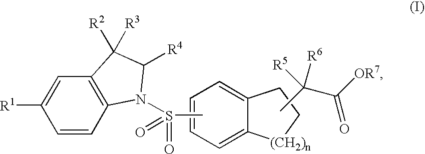

in which

R′ represents phenyl or represents 5- or 6-membered heteroaryl having up to two heteroatoms from the group consisting of N, O and S, which radicals may for their part each be mono- to trisubstituted by identical or different substituents selected from the group consisting of halogen, cyano, nitro, (C₁-C₆)-alkyl (which for its part may be substituted by hydroxyl), (C₁-C₆)-alkoxy, trifluoromethyl, trifluoromethoxy, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)alkylamino,

R² and R³ are identical or different and independently of one another represent hydrogen or (C₁-C₄)-alkyl or together with the carbon atom to which they are attached form a 3- to 7-membered spiro-linked cycloalkyl ring,

R⁴ represents hydrogen or (C₁-C₄)-alkyl,

R⁵ and R⁶ are identical or different and independently of one another represent hydrogen or (C₁-C₄)-alkyl,

R⁷ represents hydrogen or also represents a hydrolyzable group which can be degraded to the corresponding carboxylic acid,

and

n represents the number 1 or 2,

and their salts, solvates and solvates of the salts.

In the context of the invention, in the definition of R⁷, a hydrolyzable group means a group which, in particular in the body, causes the —C(O)OR⁷ grouping to be converted into the corresponding carboxylic acid (R⁷=hydrogen). Such groups are, by way of example and by way of preference: benzyl, (C₁-C₆)-alkyl or (C₃—C₈)-cycloalkyl which are in each case optionally mono- or polysubstituted by identical or different substituents from the group consisting of halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or in particular (C₁-C₄)-alkyl which is optionally mono- or polysubstituted by identical or different substituents from the group consisting of halogen, hydroxyl, amino, (C₁-C₄)-alkoxy, alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl and (C₁—C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl and tert-butyl.

In the context of the invention, (C₃-C₈)-cycloalkyl represents a monocyclic cycloalkyl group having 3 to 8 carbon atoms. The following radicals may be mentioned by way of example and by way of preference: cyclopropyl, cyclobutyl, cyclopentyl and cyclohexyl.

In the context of the invention, (C₁-C₆)-alkoxy and (C₁-C₄)alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

In the context of the invention, (C₁-C₆)-alkoxycarbonyl and (C₁-C₄)-alkoxycarbonyl represent a straight-chain or branched alkoxy radical having 1 to 6 and 1 to 4 carbon atoms, respectively, which radical is attached via a carbonyl group. Preference is given to a straight-chain or branched alkoxy-carbonyl radical having 1 to 4 carbon atoms. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, C₁-C₆)-alkoxycarbonylamino and (C₁-C₄)-alkoxycarbonylamino represent an amino group having a straight-chain or branched alkoxycarbonyl substituent which has 1 to 6 and 1 to 4 carbon atoms, respectively, in the alkoxy radical and which is attached via the carbonyl group. Preference is given to an alkoxycarbonylamino radical having 1 to 4 carbon atoms. The following radicals may be mentioned by way of example and by way of preference: methoxy-carbonylamino, ethoxycarbonylamino, n-propoxycarbonylamino and tert-butoxycarbonylamino.

In the context of the invention, (C₁-C₆)-alkanoyl represents a straight-chain or branched alkyl radical having 1 to 6 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, i-butyryl, pivaloyl and n-hexanoyl.

In the context of the invention, (C₁-C₆)-alkanoyloxy and (C₁-C₄)-alkanoyloxy represent a straight-chain or branched alkyl radical having 1 to 6 and 1 to 4 carbon atoms, respectively, which carries a doubly attached oxygen atom in the 1-position and is attached in the 1-position via a further oxygen atom. Preference is given to an alkanoyloxy radical having 1 to 4 carbon atoms. The following radicals may be mentioned by way of example and by way of preference: acetoxy, propionoxy, n-butyroxy, i-butyroxy, pivaloyloxy, n-hexanoyloxy.

In the context of the invention, mono-(C₁—C₆)-alkylamino and mono-(C₁-C₄)-alkylamino represent an amino group having a straight-chain or branched alkyl substituent of 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁—C₆)-alkylamino and di-(C₁-C₄)alkylamino represent an amino group having two identical or different straight-chain or branched alkyl substituents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₁—₆)-acylamino represents an amino group having a straight-chain or branched alkanoyl substituent which has 1 to 6 carbon atoms and is attached via the carbonyl group. Preference is given to an acylamino radical having 1 or 2 carbon atoms. The following radicals may be mentioned by way of example and by way of preference: formamido, acetamido, propionamindo, n-butyramido and pivaloylamido.

In the context of the invention, (C₁-C₆)-alkylsulphonyl represents a, straight-chain or branched alkylsulphonyl radical having 1 to 6 carbon atoms. Preference is given to a straight-chain or branched alkylsulphonyl radical having 1 to 4 carbon atoms. The following radicals may be mentioned by way of example and by way of preference: methylsulphonyl, ethylsulphonyl, n-propylsulphonyl, isopropylsulphonyl, tert-butylsulphonyl, n-pentylsulphonyl and n-hexylsulphonyl.

In the context-of the invention, 5- or 6-membered heteroaryl having up to 2 identical or different heteroatoms from the group consisting of N, O and S represents a monocyclic aromatic heterocycle (heteroaromatic) which is attached via a ring carbon atom or, if appropriate, via a ring nitrogen atom of the heteroaromatic. Examples which may be mentioned are: furanyl, pyrrolyl, thienyl, pyrazolyl, imidazolyl, thiazolyl, oxazolyl, isoxazolyl, isothiazolyl, pyridyl, pyrimidinyl, pyridazinyl, pyrazinyl. Preference is given to 5- or 6-membered heteroaryl radicals having up to two nitrogen atoms, such as, for example, imidazolyl, pyridyl, pyrimidinyl, pyridazinyl, pyrazinyl.

In the context of the invention, halogen includes fluorine, chlorine, bromine and iodine. Preference is given to chlorine or fluorine.

Depending on the substitution pattern, the compounds according to the invention can exist in stereoisomeric forms which are either like image and mirror image (enantiomers) or not like image and mirror image (diastereomers). The invention relates both to the enantiomers or diastereomers and to their respective mixtures. The racemic forms, like the diastereomers, can be separated in a known manner into the stereoisomerically uniform components.

Furthermore, certain compounds can be present in tautomeric forms. This is known to the person skilled in the art, and such compounds are likewise included in the scope of the invention.

The compounds according to the invention can also be present as salts. In the context of the invention, preference is given to physiologically acceptable salts.

Physiologically acceptable salts can be salts of the compounds according to the invention with inorganic or organic acids. Preference is given to salts with inorganic acids such as, for example, hydrochloric acid, hydrobromic acid, phosphoric acid or sulphuric acid, or to salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Physiologically acceptable salts can also be salts of the compounds according to the invention with bases, such as, for example, metal or ammonium salts. Preferred examples are alkali metal salts (for example sodium salts or potassium salts), alkaline earth metal salts (for example magnesium salts or calcium salts), and also ammonium salts which are derived from ammonia or organic amines, such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Preference is given to compounds of the general formula (I) in which

R¹ represents phenyl which may be mono- or disubstituted by identical or different substituents selected from the group consisting of fluorine, chlorine, cyano, (C₁-C₄)-alkyl, (C₁-C₄)-alkoxy, trifluoromethyl, trifluoromethoxy, methylsulphonyl, acetyl, propionyl, (C₁-C₄)-alkoxycarbonyl, amino, acetylamino, mono- and di-(C₁-C₄)-alkylamino,

R² and R³ are identical or different and independently of one another represent hydrogen or (C₁-C₄)-alkyl or together with the carbon atom to which they are attached form a 5- or 6-membered, spiro-linked cycloalkyl ring,

R⁴ represents hydrogen or methyl,

R⁵ and R⁶ are identical or different and independently of one another represent hydrogen or methyl,

R⁷ represents hydrogen,

and

n represents the number 1 or 2.

Particular preference is given to compounds of the general formula (I) in which

R¹ represents phenyl which may be mono- or disubstituted by identical or different substituents selected from the group consisting of fluorine, chlorine, methyl, trifluoromethyl and trifluoromethoxy,

R² represents methyl,

R³ represents methyl,

or

R² and R³ together with the carbon atom to which they are attached form a spiro-linked cyclopentane or cyclohexane ring,

R⁴ represents hydrogen or methyl,

R⁵ and R⁶ each represent hydrogen,

R⁷ represents hydrogen,

and

n represents the number 1 or 2.

The general or preferred radical definitions listed above apply both to the end products of the formula (I) and, correspondingly, to the starting materials and intermediates required in each case for the preparation.

The individual radical definitions given in the respective combinations or preferred combinations of radicals are, independently of the respectively given combinations of radicals, also replaced by any radical definitions of other combinations.

Very particular preference is given to combinations of two or more of the preferred ranges mentioned above.

Of particular importance are compounds of the formula (I-A)

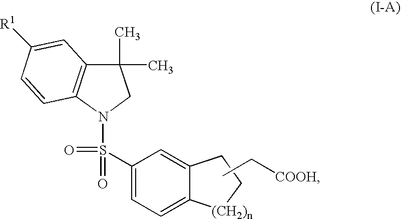

in which

R¹ represents phenyl which is substituted by fluorine, chlorine or trifluoromethyl,

and

n represents the number 1 or 2.

Moreover, we have found a process for preparing the compounds of the general, formula (I) or (I-A) according to the invention, which process is characterized in that compounds of the formula (II)

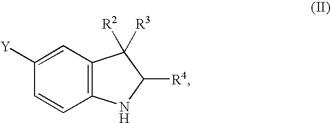

in which R², R³ and R⁴ are each as defined above and

Y represents chlorine or bromine,

are initially converted by methods known from the literature into compounds of the formula (III)

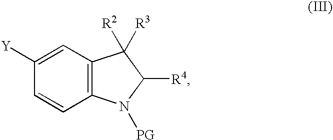

in which Y, R², R³ and R⁴ are each as defined above and

PG represents a suitable amino protective group, preferably 4-nitrophenylsulphonyl,

these compounds are then reacted in a coupling reaction with a compound of the formula (IV)

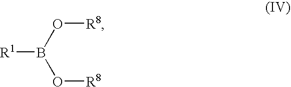

in which R¹ is as defined above and

R⁸ represents hydrogen or methyl or both radicals together form a CH₂CH₂— or C(CH₃)₂—C(CH₃)₂— bridge,

in an inert solvent in the presence of a suitable palladium catalyst and a base, to give compounds of the formula (V)

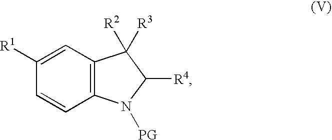

in which PG, R¹, R², R³ and R⁴ are each as defined above,

[cf. e.g. W. Hahnfeld, M. Jung, Pharmazie 1994, 49, 18-20; idem, Liebigs Ann. Chem. 1994, 59-64], the protective group PG is then removed using methods known from the literature, to give compounds of the formula (VI)

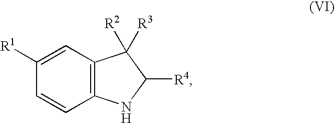

in which R¹, R², R³ and R⁴ are each as defined above,

the compounds are then, using a compound of the formula (VII)

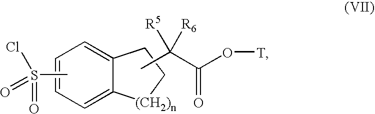

in which R⁵, R⁶ and n are each as defined above and

T represents benzyl or (C₁-C₆)alkyl,

in an inert solvent in the presence of a base converted into compounds of the formula (VIII)

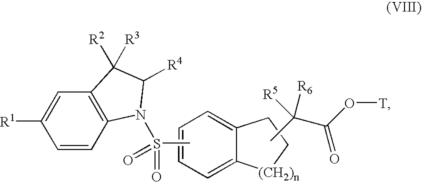

in which n, T, R¹, R², R³, R⁴, R⁵ and R⁶ are each as defined above,

the compounds of the formula (VIII) are then with acids or bases or, if T represents benzyl, also hydrogenolytically converted into the corresponding carboxylic acids of the formula (IX)

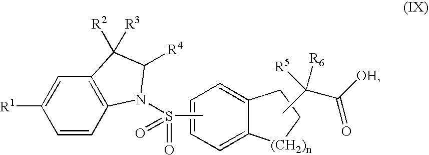

in which n, R¹, R², R³, R⁴, R⁵ and R⁶ are each as defined above,

these carboxylic acids (IX) are, if appropriate, modified further using known esterification methods to give compounds of the formula (I),

and the resulting compounds of the formula (IX) or (I) are, if appropriate, converted into their solvates, salts and/or solvates of the salts using the corresponding (i) solvents and/or (ii) bases or acids.

Inert solvents for process step (III)+(IV)→(V) are, for example, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, acetonitrile or else water. It is also possible to use mixtures of the solvents mentioned. Preference is given to toluene, dimethylformamide or acetonitrile.

Suitable bases for process step (III)+(IV)→(V) are the customary inorganic or organic bases. These preferably include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal phosphates, such as sodium phosphate or potassium phosphate, or organic amines, such as pyridine, triethylamine, ethyldiisopropylamine, N-methylmorpholine or N-methylpiperidine. Particular preference is given to sodium carbonate or potassium carbonate or potassium phosphate.

Here, the base is employed in an amount of from 1 to 5, preferably from 2 to 3, mol, based on 1 mol of the compound of the formula (III).

Suitable palladium catalysts for process step (III)+(IV)→(V) are, preferably, palladium(0) or palladium(II) compounds, which are used pre-formed, such as, for example, [1,1′-bis(diphenylphosphino)ferrocenyl]palladium(II) chloride, bis(triphenylphosphine)palladium(II) chloride or tetrakis(triphenylphosphine)palladium(0), or which may be generated in situ from a suitable palladium source, such as, for example, bis(dibenzylideneacetone)palladium(0), and a suitable phosphine ligand.

The reaction is generally carried out in a temperature range of from 0° C. to +150° C., preferably from +20° C. to +120° C. The reaction can be carried out under atmospheric, elevated or reduced pressure (for example from 0.5 to 5 bar). In general, the reaction is carried out under atmospheric pressure.

Inert solvents for process step (VI)+(VII)→(VIII) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as nitromethane, ethyl acetate, acetone, dimethylformamide, dimethyl sulphoxide, acetonitrile, N-methylpyrrolidinone or pyridine. It is also possible to use mixtures of the solvents mentioned. Preference is given to dichloromethane or tetrahydrofuran.

Suitable bases for process step (VI)+(VI)→(VIII) are the customary inorganic or organic bases. These preferably include alkali metal hydroxides, such as, for example, lithium hydroxide, sodium hydroxide or potassium hydroxide, alkali metal or alkali earth metal carbonates, such as sodium carbonate, potassium carbonate or calcium carbonate, alkali metal hydrides, such as sodium hydride, or organic amines, such as pyridine, triethylamine, ethyldiisopropylamine, N-methylmorpholine or N-methylpiperidine. Particular preference is given to amine bases, such as triethylamine, pyridine or ethyldiisopropylamine, if appropriate in the presence of catalytic amounts (about 10 mol %) of 4-N,N-dimethylaminopyridine or 4-pyrrolidinopyridine.

Here, the base is employed in an amount of from 1 to 5, preferably from 1 to 2.5, mol, based on 1 mol of the compound of the formula (VII).

The reaction is generally carried out in a temperature range of from −20° C. to +100° C., preferably from 0° C. to +75° C. The reaction can be carried out under atmospheric, elevated or reduced pressure (for example from 0.5 to 5 bar). In general, the reaction is carried out under atmospheric pressure.

Inert solvents for process step (VIII)→(IX) are, for example, halogenated hydrocarbons, such as dichloromethane, 1,2-dichloroethane or trichloroethylene, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, acetone, dimethylformamide, dimethyl sulphoxide, acetonitrile, N-methylpyrrolidinone or else water. It is also possible to use mixtures of the solvents mentioned. Preference is given to alcohols, such as methanol or ethanol, and mixtures thereof with tetrahydrofuran.

Suitable bases for process step (VIII)→(IX) are the customary inorganic bases. These preferably include alkali metal hydroxides, such as, for example, lithium hydroxide, sodium hydroxide or potassium hydroxide, or alkali metal or alkaline earth metal carbonates, such as sodium carbonate, potassium carbonate or calcium carbonate. Particular preference is given to lithium hydroxide or sodium hydroxide.

Here, the base is employed in an amount of from 1 to 5, preferably from 1 to 3, mol, based on 1 mol of the compound of the formula (VIII).

Suitable acids for process step (VIII)→(IX) are the customary inorganic acids, such as, for example, hydrochloric acid or sulphuric acid, or sulphonic acids, such as toluenesulphonic acid, methanesulphonic acid or trifluoromethanesulphonic acid, or carboxylic acids, such as trifluoroacetic acid.

The reaction is generally carried out in a temperature range of from −20° C. to +100° C., preferably from 0° C. to +30° C. The reaction can be carried out under atmospheric, elevated or reduced pressure (for example from 0.5 to 5 bar). In general, the reaction is carried out under atmospheric pressure.

The compounds of the formula (II) are known or can be prepared analogously to processses known from the literature, for example by reacting compounds of the formula (X)

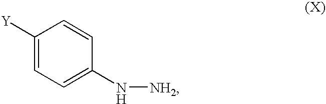

in which Y is as defined above,

in the presence of an acid or Lewis acid, if appropriate in an inert solvent, with a compound of the formula (XI)

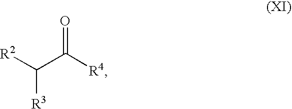

in which R², R³ and R⁴ are as defined above,

if R² and R³ in (XI) are both not hydrogen, to give compounds of the formula (XII), if R³ in (XI) is hydrogen, to give compounds of the formula (XIII)

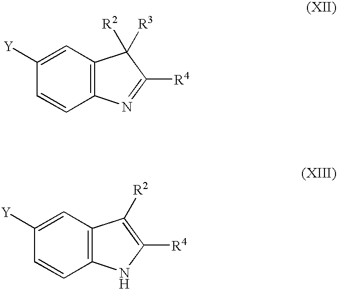

in which Y and R⁴ are each as defined above,

and then reducing the compounds of the formula (XII) or (XIII) with the aid of a boron hydride, aluminium hydride or silicon hydride, such as, for example, sodium borohydride or sodium cyano-borohydride, or by hydrogenation in the presence of a suitable catalyst, such as, for example, Raney-nickel [for process steps (X)+(XI)→(XII)→(II), cf., for example, P. E. Maligres, I. Houpis, K. Rossen, A. Molina, J. Sager, V. Upadhyay, K. M. Wells, R. A. Reamer, J. E. Lynch, D. Askin, R. P. Volante, P. J. Reider, Tetrahedron 1997, 53, 10983-10992].

Inert solvents for process step (X)+(XI)→(XII) or (XIII) are, for example, halogenated hydrocarbons, such as dichloromethane, trichloromethane, carbon tetrachloride, trichloroethane, tetrachloroethane, 1,2-dichloroethane or trichloroethylene, ethers, such as dioxane, tetrahydrofuran, glycol dimethyl ether or diethylene glycol dimethyl ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile or water. It is also possible to use mixtures of the solvents mentioned. It is also possible to carry out the reaction in the absence of a solvent. If R³ represents hydrogen, the reaction is preferably carried out in the absence of a solvent to give the product (XI), if R² and R³ are both not hydrogen, the reaction is preferably carried out in a mixture of toluene and acetonitrile to give the product (XII).

Suitable acids for process step (X)+(XI)→(XH) or (XIII) are the customary inorganic or organic acids. These preferably include hydrochloric acid, sulphuric acid or phosphoric acid, or carboxylic acids, such as formic acid, acetic acid, or trifluoroacetic acid, or sulphonic acids, such as toluenesulphonic acid, methanesulphonic acid or trifluoromethanesulphonic acid. Alternatively, the customary Lewis acids, such as, for example, boron trifluoride, aluminium trichloride or zinc chloride are also suitable. Here, the acid is employed in an amount of from 1 to 10 mol, based on 1 mol of the compound of the formula (X). If R³ represents hydrogen, the reaction is preferably carried out using 1 to 2 mol of zinc chloride to give the product (XIII), and if R² and R³ are both not hydrogen, the reaction is preferably carried out using 2 to 5 mol of trifluoroacetic acid to give the product (XII).

The reaction is generally carried out in a temperature range of from 0° C. to +250° C. If R³ represents hydrogen, the reaction is preferably carried out in a temperature range of from +130° C. to +200° C. to give the product (XIII), if R² and R³ are both not hydrogen, the reaction is preferably carried out in a temperature range of from 0° C. to +50° C. to give the product (XII). The reaction can be carried out under atmospheric, elevated or reduced pressure (for example from 0.5 to 5 bar). In general, the reaction is carried out under atmospheric pressure.

Reducing agents suitable for process step (XII) or (XIII)→(II) are boron hydrides, aluminium hydrides or silicon hydrides, such as, for example, borane, diborane, sodium borohydride, sodium cyanoborohydride, lithium aluminium hydride or triethylsilane, if appropriate in the presence of an acid or Lewis acid, such as, for example, acetic acid, trifluoroacetic acid, aluminium trichloride or boron trifluoride, or the hydrogenation with hydrogen in the presence of a suitable catalyst, such as, for example, palladium-on-carbon, platinum oxide or Raney-nickel. For compounds of the formula (XIII), preference is given to reduction using sodium cyanoborohydride; for compounds of the formula (XII), preference is given to using sodium borohydride.

Suitable solvents for process step (XII) or (XIII)→(II) are, for example, ethers, such as diethyl ether, dioxane, tetrahydrofuran, glycol dimethyl ether or diethylene glycol dimethyi ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile, acetic acid or water. It is also possible to use mixtures of the solvents mentioned. For reducing the compounds of the formula (XIII), preference is given to using acetic acid, a large excess of which, added as acid to the reducing agent, simultaneously serves as solvent. For reducing the compounds of the general formula (XII), preference is given to using a mixture of methanol and toluene/acetonitrile [from the reaction (X)→(XII), with addition of 2 to 5 mol of trifluoroacetic acid] in a ratio of from 1:1 to 1:10.

The reaction is generally carried out in a temperature range of from −20° C. to +100° C., preferably from −10° C. to +50° C. The reaction can be carried out under atmospheric, elevated or reduced pressure (for example from 0.5 to 5 bar). In general, the reaction is carried out under atmospheric pressure.

The compounds of the formula (VII) are known or can be prepared analogously to processes known from the literature, for example by initially converting compounds of the formula (XIV)

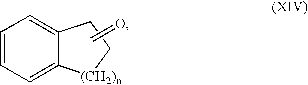

in which n is as defined above,

in a Wittig or Wittig-Horner reaction [see, for example, J. Heterocycl. Chem. 1986, 747; J. Org. Chem. 1991, 6717] or via zinc organyls [see, for example, J. Amer. Chem. Soc. 1958, 4360] into compounds of the formula (XV)

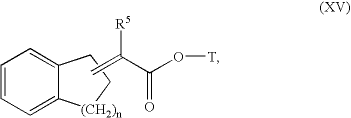

in which R⁵, n and T are each as defined above,

which are then, in the presence of a suitable catalyst, such as, for example, palladium-on-active carbon, hydrogenated to give compounds of the formula (XVI)

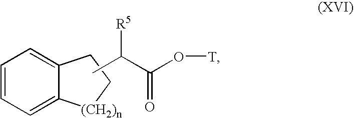

in which R⁵, n and T are each as defined above,

and finally reacted with chlorosulphonic acid [cf., for example, P. D. Edwards, R. C. Mauger, K. M. Cottrell, F. X. Morris, K. K. Pine, M. A. Sylvester, C. W. Scott, S. T. Furlong, Bioorg. Med. Chem. Lett. 2000, 10, 2291-2294].

The compounds of the formulae (IV), (X), (XI) and (XIV) are commercially available, known from the literature or can be prepared analogously to the processes known from the literature.

The process according to the invention can be illustrated by reaction schemes 1 and 2 below:

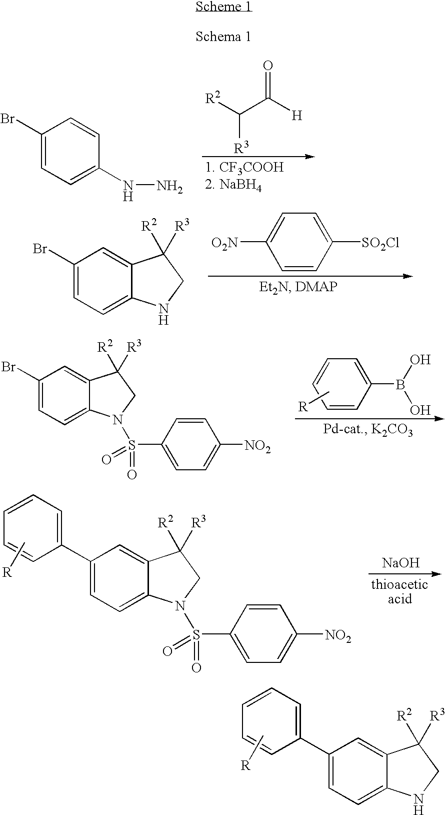

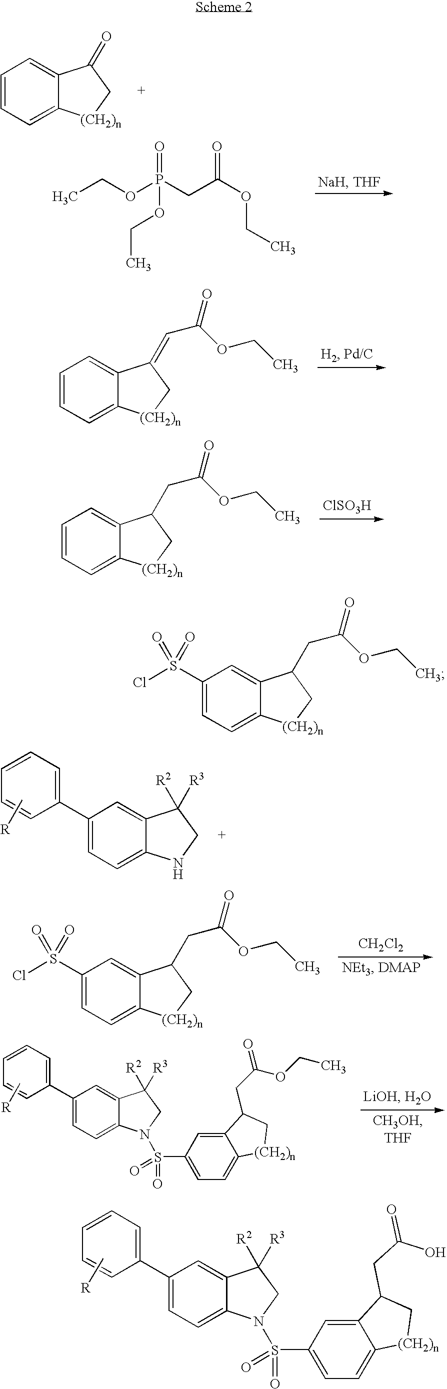

The compounds of the formula (I) according to the invention have a surprising and useful spectrum of pharmacological activity and can therefore be used as versatile medicaments. In particular, they are suitable for treating coronary heart disease, for the prophylaxis of myocardial infarction and for the treatment of restenosis after coronary angioplasty or stenting. The compounds of the formula (I) according to the invention are preferably suitable for treating arteriosclerosis and hypercholesterolaemia, for increasing pathologically low HDL levels and for lowering elevated triglyceride and LDL levels. In addition, they can be used for treating obesity, diabetes, for treating metabolic syndrome (glucose intolerance, hyperinsulinaemia, dyslipidaemia and high blood pressure owing to insulin resistance), hepatic fibrosis and cancer.

The novel active compounds can be administered alone or, if required, in combination with other active compounds, preferably from the group of the CETP inhibitors, antidiabetics, antioxidants, cytostatics, calcium antagonists, antihypertensives, thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol absorption inhibitors, MTP inhibitors, aldolase reductase inhibitors, fibrates, niacin, anorectics, lipase inhibitors and PPAR-α and/or PPAR-γ agonists.

The activity of the compounds according to the invention can be examined, for example, in vitro by the transactivation assay described in the experimental section.

The activity of the compounds according to the invention in vivo can be examined, for example, by the tests described in the experimental section.

Suitable administration forms for administering the compounds of the general formula (I) are all customary administration forms, i.e. oral, parenteral, inhalative, nasal, sublingual, rectal, external, for example transdermal, or local, such as, for example, in the case of implants or stents. In the case of parenteral administration, particular mention has to be made of intravenous, intramuscular and subcutaneous administration, for example as a subcutaneous depot. Preference is given to oral or parenteral administration. Very particular preference is given to oral administration.

Here, the active compounds can be administered on their own or in the form of preparations. Preparations suitable for oral administration are, inter alia, tablets, capsules, pellets, sugar-coated tablets, pills, granules, solid and liquid aerosols, syrups, emulsions, suspensions and solutions.

Here, the active compound has to be present in such an amount that a therapeutic effect is obtained. In general, the active compound can be present in a concentration of from 0.1 to 100% by weight, in particular from 0.5 to 90% by weight, preferably from 5 to 80% by weight. In particular, the concentration of active compound should be 0.5 to 90% by weight, i.e. the active compound should be present in amounts sufficient to reach the dosage range stated.

To this end, the active compounds can be converted in a manner known per se into the customary preparations. This is carried out using inert non-toxic pharmaceutically acceptable carriers, auxiliaries, solvents, vehicles, emulsifiers and/or dispersants.

Auxiliaries which may be mentioned are, for example: water, non-toxic organic solvents, such as, for example, paraffins, vegetable oils (for example sesame oil), alcohols (for example ethanol, glycerol), glycols (for example polyethylene glycol), solid carriers, such as natural or synthetic ground minerals (for example talc or silicates), sugar (for example lactose), emulsifiers, dispersants (for example polyvinylpyrrolidone) and glidants (for example magnesium sulphate).

In the case of oral administration, tablets may, of course, also contain additives such as sodium citrate, together with additives such as starch, gelatine and the like. Aqueous preparations for oral administration may furthermore comprise flavour improvers or colorants.

In the case of oral administration, preference is given to administering dosages of from 0.001 to 5 mg/kg, preferably from 0.005 to 3 mg/kg, of body weight per 24 hours.

The working examples below illustrate the invention. The invention is not limited to the examples.

The percentages in the tests and examples below are, unless indicated otherwise, percentages by weight; parts are weights by weight. Solvent ratios, dilution ratios and concentrations of liquid/liquid solutions are in each case based on volume.

### A. EXAMPLES

Abbreviations used:

| | |
|---|---|
| CI | chemical ionization (in MS) |
| TLC | thin-layer chromatography |
| DCI | Direct chemical ionization (in MS) |
| DMAP | 4-N,N-dimethylaminopyridine |
| DMF | N,N-dimethylformamide |
| DMSO | dimethyl sulphoxide |
| EI | electron impact ionization (in MS) |
| ESI | electrospray ionization (in MS) |
| Et | Ethyl |
| GC | gas chromatography |
| h | hour(s) |
| HPLC | high pressure, high performance liquid chromatography |
| min | minute(s) |
| MS | mass spectroscopy |
| NMR | nuclear magnetic resonance spectroscopy |
| R_{f} | retention index (in TLC) |
| RT | Room temperature |
| THF | tetrahydrofuran |

GC-MS method:

Instrument: Micromass GCT, GC6890; column: Restek RTX-35MS, 30 m×250 μm×0.25 μm; constant flow with helium: 0.88 ml/min; oven: 60° C.; inlet: 250° C.; gradient: 60° C. (maintained for 0.30 min), 50° C./min→120° C., 16° C/min→250° C., 30° C./min→300° C. (maintained for 1.7 min).

### WORKING EXAMPLES

### Example 1

[7-({3,3-Dimethyl-5-[4-(trifluoromethyl)phenyl]-2,3-dihydro-1H-indol-1-yl}sulphonyl)-1,2,3,4-tetrahydro-1-naphthalenyl]acetic acid

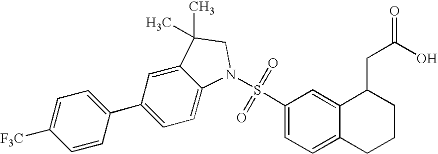

Step a):

5-Bromo-3,3-dimethylindoline

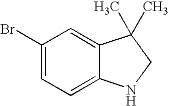

A mixture of 45 ml of toluene/acetonitrile (49:1) is flushed with argon for 5 minutes, and 3.00 g (13.4 mmol) of 4-bromophenylhydrazine are then added. 3.71 ml (48.1 mmol) of trifluoroacetic acid are then added slowly, and it is ensured that the temperature does not exceed 35° C. Subsequently, the temperature is maintained at 35° C., and a solution of 1.05 g (14.6 mmol) of isobutyraldehyde in 4 ml of toluene/acetonitrile (49:1) is slowly added dropwise. The mixture is stirred at 35° C. for 4 h and at room temperature for 2 h. The mixture is then cooled to −10° C., 4.0 ml of methanol are added and 819 mg (21.7 mmol) of solid-sodium borohydride are added a little at a time over a period of 30 min. During the addition, the temperature must not exceed −2° C. After the addition has ended, the mixture is stirred at 0° C. for 1 h. After addition of 150 ml of a 6% strength by weight solution of ammonia in water, the phases are separated, and in each case 1.5 ml of acetonitrile and methanol are added to the organic phase. The organic phase is then washed with 150 ml of a 15% strength solution of sodium chloride in water and dried over sodium sulphate. The mixture is filtered through 100 g of silica gel, and the silica gel is washed twice with in each case 200 ml of diethyl ether. The organic filtrate is concentrated under reduced pressure and chromatographed on 100 g of silica gel. Initially, the by-products are eluted with cyclohexane, subsequently the product is eluted using a mixture of cyclohexane/diethyl ether (20: 1). This gives 1.78 g (54% of theory) of the product as an oil.

R_{f} (petroleum ether/ethyl acetate 5:1)=0.47

MS (ESIpos): m/z=226 [M+H]⁺

¹H-NMR (200 MHz, DMSO-d₆): δ=1.20 (s, 6H), 3.18 (d, 2H), 5.66 (broad s, 1H), 6.42 (d, 1H), 7.02 (dd, 1H), 7.10 (d, 1H).

Step b):

5-Bromo-3,3-dimethyl-1-4-nitrophenylsulphonyl)-2,3-dihydro-1H-indole

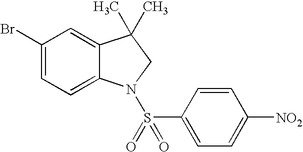

17 g (75.18 mmol) of the bromoindoline from step a), 5.22 g (150.37 mmol) of triethylamine and 0.46 g (3.76 mmol) of DMAP are dissolved in 100 ml of dichloromethane and cooled to 5-10° C. A solution of 17.5 g (78.94 mmol) of 4-nitrobenzenesulphonyl chloride in 150 ml of dichloromethane is added dropwise, and the mixture is stirred at RT for 16 h. The mixture is washed in each case once with 2 M hydrochloric acid, water and saturated sodium chloride solution, and the organic phase is dried over sodium sulphate. Removal of the solvent gives 31 g (98% of theory) of the product as a yellow solid.

MS (Cl): m/z=430 [M+NH₄]⁺

¹H-NMR (200 MHz, CDCl₃): δ=8.32 (d, 2H), 8.0 (d, 2H), 7.51 (d, 1H), 7.34 (d, 1H), 7.15 (d, 1H), 3.66 (s, 2H), 1.13 (s, 6H).

Step c):

3,3-Dimethyl-1-(4-nitrophenylsulphonyl)-5-[4-(trifluoromethyl)phenyl]-2,3-dihydro-1H-indole

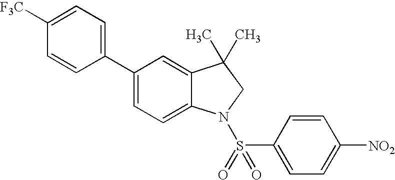

31 g (75.38 mmol) of the protected bromoindoline from step b), 21.47 g (113.06 mmol) of 4-(trifluoromethyl)phenylboronic acid and 15.63 g (113.06 mmol) of potassium carbonate are suspended in 500 ml of toluene. For 30 min, argon is passed through the solution, and 1.74 g (1.51 mmol) of tetrakis(triphenylphosphine)palladium(0) are then added. The mixture is heated under reflux for 16 h and then cooled and filtered through an about 1000 ml column with silica gel 60. Elution is initially carried out using about 1.5 l of cyclohexane and then using about 2 l of dichloromethane. The dichloromethane phase is concentrated. This gives 30 g (84% of theory) of the product as a yellow solid.

MS (EI): m/z=475.9 [M]⁺

¹H-NMR (200 MHz, CDCl₃): δ=8.32 (d, 2H), 8.05 (d, 2H), 7.71 (d, 1H), 7.66 (d, 2H), 7.61 (d, 2H), 7.46 (dd, 1H), 7.26 (s, 1H), 3.73 (s, 2H), 1.21 (s, 6H).

Step d):

3,3-Dimethyl-5-[4-(trifluoromethyl)phenyl]-2,3-dihydro-1H-indole

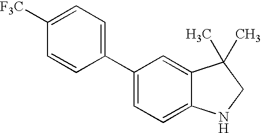

At RT, 68 g (142.72 mmol) of the indoline derivative from step c), together with 25.12 g (0.628 mol) of sodium hydroxide, are initially charged in 300 ml of DMF. 28.92 g (0.314 mol) of thioacetic acid are quickly added dropwise, and the reaction mixture is heated at 45° C. for 5 h. 1000 ml of ethyl acetate are added, and the mixture is washed twice with sodium carbonate solution and once with saturated sodium chloride solution. The organic phase is dried over sodium sulphate and concentrated. The residue is filtered through silica gel 60 (1 kg) using the mobile phase cyclohexane/ethyl acetate (7:1). This gives 27.1 g (61% of theory) of the product as a solid of light-yellow colour.

MS (EI): m/z=292.1 [M]⁺

¹H-NMR (200 MHz, CDCl₃): δ=7.62 (s, 4H), 7.31 (d, 1H), 7.27 (m, 2H), 6.69 (d, 1H), 3.39 (s, 2H), 1.36 (s, 6H).

Step e):

Ethyl 3,4-dihydro-1(2H)-naphthalenylideneethanoate

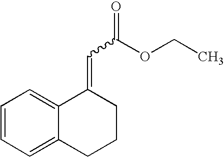

13 g (40.22 mmol) of sodium ethoxide are initially charged in a 100 ml round-bottomed flask, and 6.7 ml (33.5 mmol) of triethyl phosphonoacetate are added dropwise. 10 ml of anhydrous ethanol are added, and after 5 min of stirring, 5 g (33.5 mmol) of 1-tetralone are slowly added dropwise over a period of 10 min. The dark-brown solution is heated under reflux for 18 h. The solvent is removed completely and the residue is purified by flash chromatography on silica gel (mobile phase: cyclohexane/ ethyl acetate 30+1→7+1). The resulting product (mixture of the E/Z isomers) which is still slightly contaminated with 1-tetralone, is hydrogenated without further purification, in the manner described below.

Step f):

Ethyl 1,2,3,4-tetrahydro-1-naphthalenylacetate

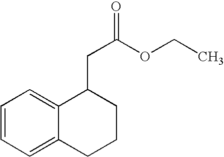

4.2 g (19.4 mmol) of the unsaturated ester from step e) are dissolved in 300 ml of ethanol, 2 g of palladium-on-active carbon (10%) are added and the mixture is hydrogenated at a hydrogen pressure of 3 bar for 4 h. The catalyst is filtered off and the solvent is removed. The residue is subjected to fractional distillation under oil pump vacuum, and the fractions are analyzed by GC-MS. The fraction which distills at 160-170° C. and about 1 mbar has a content of 99% of the desired product.

Yield: 0.7 g (17% of theory)

GC-MS (CI): m/z=219 [M+H]⁺

¹H-NMR (200 MHz, DMSO-d₆): δ=7.18-6.99 (m, 4H), 4.09 (q, 2H), 3.20 (m, 1H), 2.75-2.65 (m, 2H), 2.53-2.40 (m, 2H), 1.85-1.54 (m, 4H), 1.89 (t, 3H).

Step g):

Ethyl [7-(chlorosulphonyl-1,2,3,4-tetrahydro-1-naphthalenyl]acetate

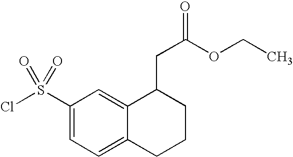

At 0° C., 393 mg (1.787 mmol) of the tetrahydronaphthalene derivative from step f) are initially charged in 13 ml of trichloromethane, and 831 mg (7.13 mmol) of chlorosulphonic acid are added dropwise. The mixture is stirred at RT for 3 h, ice is then added, and the mixture is extracted three times with ethyl acetate. The combined organic phases are dried over magnesium sulphate and the solvent is removed completely. The resulting crude product is used for the next step without further purification.

Yield: 292 mg (52% of theory)

MS (DCI, NH₃): m/z=333 [M+NH₃+NH₄]⁺

¹H-NMR (300 MHz, CDCl₃): δ=7.69 (s, 1H), 7.60 (d, 1H), 7.12 (d, 1H), 4.14 (q, 2H), 3.33-3.24 (m, 1H), 2.89-2.48 (m, 4H), 1.90-1.65 (m, 4H), 1.24 (t, 3H).

Step h):

Ethyl [7-({3,3-dimethyl-5-[4-(trifluoromethyl)phenyl]-2,3-dihydro-1H-indol-1-yl }sulphonyl)-1,2,3,4-tetrahydro-1-naphthalenyl]acetate

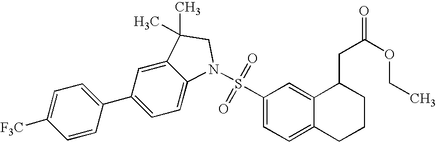

94 mg (0.324 mmol) of the indoline derivative from step d) are initially charged in 3 ml of dichloromethane. 0.052 ml (0.65 mmol) of triethylamine and 4 mg (0.032 mmol) of DMAP are added. At RT, a solution of 113 mg (0.357 mmol) of the sulphonyl chloride from step g) in 2 ml of dichloromethane is slowly added dropwise, and the mixture is stirred at RT for 5 h. The solvent is removed completely and the residue is purified by preparative HPLC. The product is isolated as a yellow solid.

Yield: 129 mg (63% of theory)

MS (DCI, NH₃): m/z=589 [M+NH₄]⁺

¹H-NMR (500 MHz, CDCl₃): δ=7.70 (d, 1H), 7.66-7.61 (m, 4H), 7.56 (d, 1H), 7.45 (d, 1H), 7.23 (s, 2H), 7.15 (d, 1H), 4.13 (m, 2H), 3.68 (q, 2H), 3.34 (m, 1H), 2.82-2.72 (m, 2H), 2.54-2.41 (m, 2H), 1.93-1.62 (m, 4H), 1.24 (t, 3H), 1.23 (s, 3H), 1.17 (s, 3H).

Step i):

[7-({3,3-Dimethyl-5-[4-(trifluoromethyl)phenyl]-2,3-dihydro-1H-indol-1-yl}sulphonyl)-1,2,3,4-tetrahydro-1-naphthalenyl]acetic acid

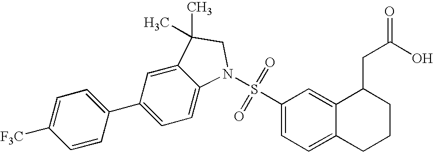

100 mg (0.175 mmol) of the ester derivative from step h) are dissolved in 1 ml of THF and 1 ml of methanol, and a solution of 8.4 mg (0.35 mmol) of lithium hydroxide in 0.4 ml of water is then added. The mixture is stirred at 50° C. for 1 h, and the cooled solution is then adjusted to pH 5 using 1 M hydrochloric acid. The mixture is extracted three times with ethyl acetate, the combined organic phases are dried over sodium sulphate and the solvent is removed completely. The residue is purified by preparative HPLC.

Yield: 45 mg (38% of theory)

MS (DCI, NH₃): m/z=561 [M+NH₄]⁺

¹H-NMR (300 MHz, DMSO-d₆): δ=12.27 (broad s, 1H), 7.83 (d, 2H), 7.75 (d, 2H), 7.72 (d, 1H), 7.61-7.50 (m, 4H), 7.26 (d, 1H), 3.71 (s, 2H), 3.22 (m, 1H), 2.73 (dd, 2H), 2.57-2.34 (m, 2H), 1.88-1.53 (m, 4H), 1.15 (s, 6H).

### Example 2

[6-({3,3-Dimethyl-5-[4-(trifluoromethyl)phenyl]-2,3-dihydro-1H-indol-1-yl}sulphonyl)-2,3-dihydro-1H-inden-1-yl]acetic acid

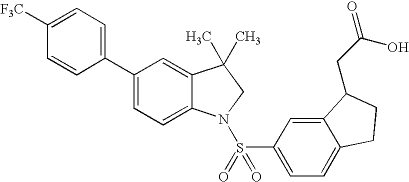

Step a):

Ethyl 2,3-dihydro-1H-inden-1 -ylideneethanoate

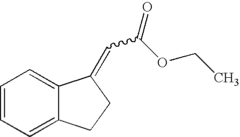

In a 100 ml round-bottom flask, 19.9 g (88.9 mmol) of triethylphosphonoacetate are initially charged in 30 ml of THF, and 3.9 g (97.9 mmol) of sodium hydride (60% in mineral oil) are added a little at a time. During the addition, the temperature should not exceed 30° C. After the addition has ended, the mixture is stirred for 10 min, and 4 g (29.7 mmol) of 1-indanone are then added. The solution is heated under reflux for 18 h. After cooling, water is added and the mixture is extracted three times with ethyl acetate. The combined organic phases are washed once with sodium chloride solution and then dried over sodium sulphate. After removal of the solvent, the crude product is chromatographed on silica gel (mobile phase: cyclohexane/ethyl acetate 1+0→10+1). This gives 5.53 g (70% of theory) of the product (as a mixture of the E/Z isomers) which, according to GC-MS, has a purity of about 76%.

MS (DCI, NH₃): m/z=220 [M+NH₄]⁺.

Step b):

Ethyl 2,3-dihydro-1H-inden-1-ylacetate

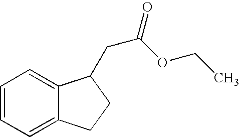

Analogously to the procedure of Example 1, step f), 1.88 g (9.34 mmol) of the unsaturated ester from step a) are hydrogenated in the presence of 10% palladium-on-carbon.

Yield: 1.84 g (90% of theory)

GC-MS (CI): m/z=222 [M+NH₄]⁺

¹H-NMR (200 MHz, CDCl₃): δ=7.35-7.09 (m, 4H), 4.18 (q, 2H), 3.59 (m, 1H), 3.0-2.70 (m, 3H), 2.50-2.30 (m, 2H), 1.85-1.65 (m, 1H), 1.28 (t, 3H).

Step c):

Ethyl [6-(chlorosulphonyl)-2,3-dihydro-1H-inden-1-yl]acetate

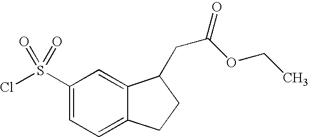

1.4 g (12.24 mmol) of chlorosulphonic acid are initially charged in 15 ml of chloroform and heated to 70° C. At this temperature, a solution of 500 mg (2.45 mmol) of the indane derivative from step b) is added dropwise, and the mixture is stirred for 30 min. After cooling, water is added and the mixture is extracted three times with ethyl acetate. The combined organic phases are dried over magnesium sulphate. After removal of the solvent, the crude product is used without further purification for forming the sulphonamide.

Yield: 437 mg (59% of theory)

MS (DCI, NH₃): m/z=320 [M+NH₄]⁺.

Step d):

Ethyl [6-({3,3-dimethyl-5-[4-(trifluoromethyl)phenyl]-2,3-dihydro-1H-indol-1-yl}sulphonyl)-2,3-dihydro-1H-inden-1-yl]acetate

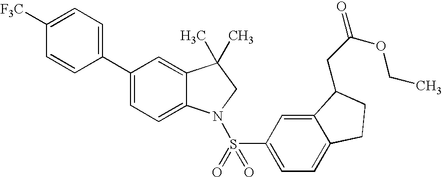

At RT, 44 mg (0.15 mmol) of the indoline derivative from Example 1, step d), are dissolved in 2 ml of anhydrous dichloromethane, and 0.024 ml (0.3 mmol) of triethylamine and 1.8 mg (0.015 mmol) of DMAP are added. A solution of 50 mg (0.165 mmol) of the sulphonyl chloride from step c) in 2 ml of dichloromethane is added dropwise, and the mixture is stirred at RT for 24 h. The water and ethyl acetate are added, and the aqueous phase is re-extracted three times with ethyl acetate. The combined organic phases are dried over sodium sulphate and the solvent is removed. The residue is purified by preparative HPLC.

Yield: 17 mg (20% of theory)

MS (ESIpos): m/z=558 [M+H]⁺

1H-NMR (500 MHz, CDCl₃): δ=7.73-7.59 (m, 7H), 7.44 (d, 1H), 7.32-7.20 (m, 2H), 4.15 (q, 2H), 3.69 (s, 2H), 3.59 (m, 1H), 3.02-2.82 (m, 2H), 2.70 (dd, 1H), 2.45-2.33 (m, 2H), 1.76 (m, 1H), 1.26 (t, 3H), 1.21 (s, 3H), 1.20 (s, 3H).

Step e):

[6-({3,3-Dimethyl-5-[4-(trifluoromethyl)phenyl]-2,3-dihydro-1H-indol-1-yl}sulphonyl)-2,3-dihydro-1H-inden-1-yl]acetic acid

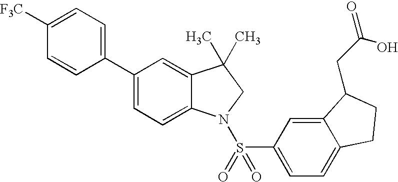

16 mg (0.029 mmol) of the ester derivative from step d) are dissolved in 0.5 ml of THF and 0.5 ml of methanol, and a solution of 2 mg (0.08 mmol) of lithium hydroxide in 0.1 ml of water is then added. The mixture is stirred at 50° C. for 1 h, and the cooled solution is then adjusted to pH 5 using 1 M hydrochloric acid. The mixture is extracted three times with ethyl acetate, the combined organic phases are dried over sodium sulphate and the solvent is removed completely.

Yield: 14 mg (91% of theory)

MS (DCI, NH₃): m/z=547 [M+NH₄]⁺

¹H-NMR (300 MHz, CDCl₃): δ=7.74-7.57 (m, 7H), 7.45 (dd, 1H), 7.33-7.21 (m, 2H), 3.70 (d, 2H), 3.62 (m, 1H), 3.0-2.88 (m, 2H), 2.73 (dd, 1H), 2.52-2.40 (m, 2H), 1.81 (m, 1H), 1.22 (s, 3H), 1.18 (s, 3H).

### Example 3

[5-({3,3-Dimethyl-5-[4-(trifluoromethyl)phenyl]-2,3-dihydro-1H-indol-1-yl }sulphonyl)2,3-dihydro-1H-inden-2-yl]acetic acid

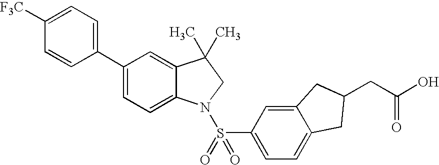

Step a):

Ethyl 1,3-dihydro-2H-inden-2-ylideneacetate

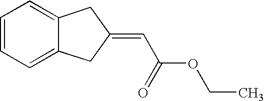

In a 100 ml round-bottom flask, 31.2 g (136.2 mmol) of triethyl phosphonoacetate are initially charged in 225 ml of THF, and 5.99 g (149.8 mmol) of sodium hydride (60% in mineral oil) are added a little at a time. During the addition, the temperature should not exceed 30° C. After the addition has ended, the mixture is stirred for 10 min and 6 g (45.4 mmol) of 2-indanone are then added. The solution is heated under reflux for 18 h. After cooling, water is added and the mixture is extracted three times with ethyl acetate. The combined organic phases are washed once with sodium chloride solution and then dried over sodium sulphate. After removal of the solvent, the crude product is chromatographed on silica gel (mobile phase: cyclohexane/ethyl acetate 10+1).

Yield: 2.83 g (24% of theory)

MS (EIpos): m/z=202 [M]⁺

¹H-NMR (300 MHz, CDCl₃): δ=7.39 (d, 1H), 7.30 (d, 1H), 7.22 (m, 1H), 7.14 (dt, 1H), 6.69 (s, 1H), 4.17 (q, 2H), 3.51 (s, 2H), 3.45 (s, 2H), 1.28 (t, 3H).

Step b):

Ethyl 2,3-dihydro-1H-inden-2-ylacetate

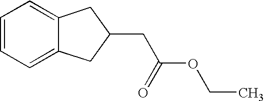

Analogously to the procedure of Example 1, step f), 2.83 g (14 mmol) of the unsaturated ester from step a) are hydrogenated in the presence of 10% palladium-on-carbon.

Yield: 2.66 g (93% of theory)

MS (EIpos): m/z=204 [M]⁺

¹H-NMR (300 MHz, CDCl₃): δ=7.22-7.09 (m, 4H), 4.15 (q, 2H), 3.13 (dd, 2H), 2.89 (quin, 1H), 2.65 (dd, 2H), 2.48 (d, 2H), 1.27 (t, 3H).

Step c):

Ethyl [5-(chlorosulphonyl)-2,3-dihydro-1H-inden-2-yl]acetate

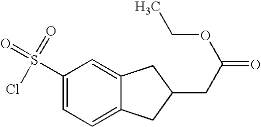

At 0° C., 2.65 g (12.97 mmol) of the indane derivative from step b) are initially charged in trichloromethane, and 15.12 g (0.137 mol) of chlorosulphonic acid are slowly added dropwise. The reaction mixture is stirred at RT for 2 h and then poured onto ice. The mixture is extracted three times with ethyl acetate, and the combined organic phases are dried over sodium sulphate. After removal of the solvent, the crude product is used without further purification for forming the sulphonamide.

Yield: 3.56 g (55% of theory)

MS (DCI, NH₃): m/z=320 [M+NH₄]⁺

¹H-NMR (300 MHz, CDCl₃): δ=7.22-7.09 (m, 3H), 4.15 (q, 2H), 3.13 (dd, 2H), 2.89 (quin, 1H), 2.65 (dd, 2H), 2.48 (d, 2H), 1.27 (t, 3H).

Step d):

Ethyl [5-({3,3-dimethyl-5-[4-(trifluoromethyl)phenyl]-2,3-dihydro-1H-indol-1-yl }sulphonyl)-2,3-dihydro-1H-inden-2-yl]acetate

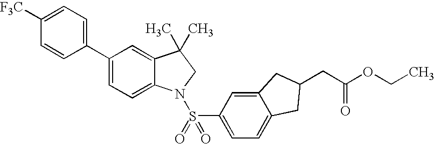

Analogously to the procedure of Example 2, step d), 120 mg (40% of theory) of the title compound are prepared from 150 mg (0.515 mmol) of the indoline derivative from Example 1, step d), by reaction with 311 mg (1.03 mmol) of the sulphonyl chloride from step c).

MS (DCI, NH₃): m/z=575 [M+NH₄]⁺

¹H-NMR (300 MHz, CDCl₃): δ=7.72-7.58 (m, 7H), 7.43 (dd, 1H), 7.28-7.22 (m, 2H), 4.12 (q, 2H), 3.70 (s, 2H), 3.15 (dd, 2H), 2.90 (quin, 1H), 2.66 (dd, 2H), 2.45 (d, 2H), 1.26 (s, 3H), 1.25 (s, 3H), 1.23 (t, 3H).

Step e):

[5-({3,3-Dimethyl-5-[4-(trifluoromethyl)phenyl]-2,3-dihydro-1H-indol-1-yl}sulphonyl)-2,3-dihydro-1H-inden-2-yl]acetic acid

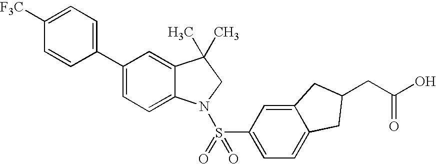

100 mg (0.179 mmol) of the ester derivative from step d) are dissolved in 5 ml of THF and 5 ml of methanol, and a solution of 9 mg (0.359 mmol) of lithium hydroxide in 1 ml of water is then added. The mixture is stirred at 50° C. for 16 h, and the cooled solution is then adjusted to pH 5 using 2 M hydrochloric acid. The solvent is removed completely, and the residue is purified by preparative HPLC.

Yield: 90 mg (95% of theory)

¹H-NMR (300 MHz, DMSO-d₆): δ=12.06 (broad s, 1H), 7.85 (d, 2H), 7.76 (d, 2H), 7.72 (s, 1H), 7.66-7.52 (m, 4H), 7.39 (d, 1H), 3.71 (s, 2H), 3.09 (m, 2H), 2.77-2.59 (m, 3H), 2.37 (d, 2H), 1.99 (s,6H).

B. Assessment of the Physiological Activity

### Example A

Cellular Transactivation Assay:

Test Principle:

A cellular assay is used to identify activators of the peroxisome proliferator-activated receptor

delta (PPAR-delta). Since mammalian cells contain different endogenous nuclear receptors which may complicate an unambiguous interpretation of the results, an established chimera system is used in which the ligand binding domain of the human PPARδ receptor is fused to the DNA binding domain of the yeast transcription factor GAL4. The resulting GAL4-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARδ.

Transactivation Assay (Luciferase Reporter):

CHO (chinese hamster ovary) cells are sown in CHO-A-SFM medium (GIBCO), supplemented by 2.5% foetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in the abovementioned medium and added to the cells. After a stimulation period of 24 hours, the luciferase activity is measured using a video camera. The relative light units measured give, as a function of the substance concentration, a sigmoidal stimulation curve. The EC₅₀ values are calculated using the computer program GraphPad PRISM (Version 3.02).

In this test, Working Examples 1-3 show EC₅₀ values in a range of from 10 to 100 nM.

### Example B

Description of the test for finding pharmacologically active substances which increase HDL cholesterol (HDL-C) concentrations in the serum of transgenic mice transfected with the human ApoA1 gene (hApoA1) and/or have an effect on the metabolic syndrome of adipose ob,ob mice and lower their blood glucose concentration:

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoAl, serum cholesterol, HDL-C and serum triglycerides (TG) (zero value). Subsequently, using a stomach tube, the test substance is administered for the first time to the animals. 24 hours after the last administration of substance (i.e. on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Geraitebau, Netheler & Hinz GmbH, Hamburg). The said determinations are carried out using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse-ApoA1 is determined with a Sandwich ELISA method using a polyclonal anti-human-ApoA1 antibody and a monoclonal anti-human-ApoA1 antibody (Biodesign International, USA). Quantification is carried out UV-photometrically (BIO-TEK Instruments, USA) using peroxidase-coupled anti-mouse-IGG antibodies (KPL, USA) and peroxidase substrate (KPL, USA).

The effect of the test substances on the HDL-C concentration is determined by subtracting the value measured for the 1st blood sample (zero value) from the value measured for the 2nd blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to that of the control group, are considered to be pharmacologically effective.

To examine substances for their effect on a metabolic syndrome, animals having an insulin resistance and increased blood glucose levels are used. To this end, C57Bl/6J Lep <ob> mice are treated using the same protocol as for the transgenic ApoA1 mice. The serum lipids are determined as described above. In these animals, serum glucose is additionally determined, as a parameter for blood glucose. Serum glucose is determined enzymatically in an EPOS Analyzer 5060 (see above), using commercially available enzyme tests (Boehringer Mannheim).

A blood-glucose-lowering effect of the test substances is determined by subtracting the value measured for the 1st blood sample of an animal (zero value) from the value measured for the 2nd blood sample of the same animal (after the treatment). The mean of the differences of all serum glucose values of one group is determined and compared to the mean of the differences of the control group.

Statistical evaluation is carried out using Student's t-test, after the variances have been checked for homogeneity.

Substances which lower the serum glucose concentration of the treated animals in a statistically significant (p<0.05) manner by at least 10%, compared to the control group, are considered to be pharmacologically effective.

C. Working Examples of Pharmaceutical Compositions

The compounds according to the invention can be converted into pharmaceutical preparations in the following way:

Tablet:

Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for format of the tablet). A compressive force of 15 kN is used as guideline for the compression.

Suspension Which can be Administered Orally:

Composition:

1000 mg of the compound of Example 1, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the compound according to the invention is added to the suspension. The water is added while stirring. The mixture is stirred for about 6 h until the swelling of the Rhodigel is complete.

Solution Which can be Administered Orally:

Composition:

500 mg of the compound of Example 1, 2.5 g of polysorbate and 97 g of polyethylene glycol 400. 20 g of oral solution corresponds to a single dose of 100 mg of the compound according to the invention.

Production:

The compound according to the invention is suspended with stirring in the mixture of polyethylene glycol and polysorbate. Stirring is continued until the compound according to the invention is completely dissolved.

1. A compound of formula (I) 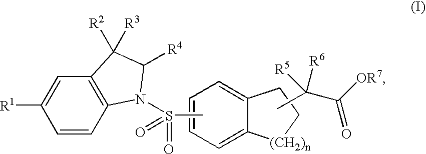
in which
R¹ represents phenyl or represents 5- or 6-membered heteroaryl having up to two heteroatoms from the group consisting of N, O and S, which radicals may for their part each be mono- to trisubstituted by identical or different substituents selected from the group consisting of halogen, cyano, nitro, (C₁-C₆)-alkyl (which for its part may be substituted by hydroxyl), (C₁-C₆)-alkoxy, trifluoromethyl, trifluoro-methoxy, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)-alkylamino,
R² and R³ are identical or different and independently of one another represent hydrogen or (C₁-C₄)-alkyl or together with the carbon atom to which they are attached form a 3- to 7-membered spiro-linked cycloalkyl ring,
R⁴ represents hydrogen or (C₁-C₄)-alkyl,
R⁵ and R⁶ are identical or different and independently of one another represent hydrogen or (C₁-C₄)-alkyl,
R⁷ represents hydrogen or also represents a hydrolyzable group which can be degraded to the corresponding carboxylic acid,
and
n represents the number 1 or 2,
or a pharmaceutically acceptable salt thereof.

2. The compound of claim 1 in which
R¹ represents phenyl which may be mono- or disubstituted by identical or different substituents selected from the group consisting of fluorine, chlorine, cyano, (C₁-C₄)-alkyl, (C₁-C₄)-alkoxy, trifluoromethyl, trifluoromethoxy, methylsulphonyl, acetyl, propionyl, (C₁-C₄)-alkoxycarbonyl, amino, acetylamino, mono- and di-(C₁-C₄)-alkylamino,
R² and R³ are identical or different and independently of one another represent hydrogen or (C₁-C₄)-alkyl or together with the carbon atom to which they are attached form a 5- or 6-membered, spiro-linked cycloalkyl ring,
R⁴ represents hydrogen or methyl,
R⁵ and R⁶ are identical or different and independently of one another represent hydrogen or methyl,
R⁷ represents hydrogen, and
n represents the number 1 or 2.

3. The compound of claim 1, in which
R¹ represents phenyl which may be mono- or disubstituted by identical or different substituents selected from the group consisting of fluorine, chlorine, methyl, tri-fluoromethyl and trifluoromethoxy,
R² represents methyl,
R³ represents methyl, or
R² and R³ together with the carbon atom to which they are attached form a spiro-linked cyclopentane or cyclohexane ring,
R⁴ represents hydrogen or methyl,
R⁵ and R⁶ each represent hydrogen,
R⁷ represents hydrogen, and
n represents the number 1 or 2.

4. A compound of formula (I-A) 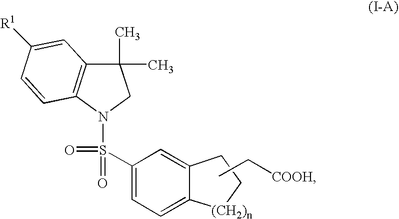
in which
R¹ represents phenyl which is substituted by fluorine, chlorine or trifluoromethyl, and
n represents the number 1 or 2.

5. A process for preparing a compound of claim 1 or claim 4 comprising initially converting a compound of the formula (II) 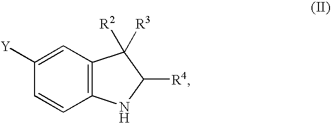
in which R², R³ and R⁴ are each as defined in claim 1 and Y represents chlorine or bromine,
by methods known from the literature into a compound of the formula (III) 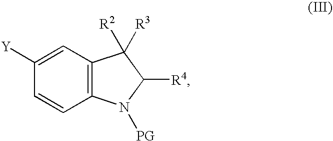
in which Y, R², R³ and R⁴ are each as defined in claim 1 and PG represents a suitable amino protective group, then reacting this compound in a coupling reaction with a compound of the formula (IV) 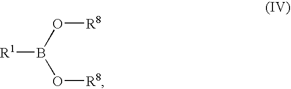
in which R¹ is as defined in claim 1 and
R⁸ represents hydrogen or methyl or both radicals together form a CH₂CH₂— or C(CH₃)₂—C(CH₃)₂— bridge,
in an inert solvent in the presence of a suitable palladium catalyst and a base, to give a compound of the formula (V) 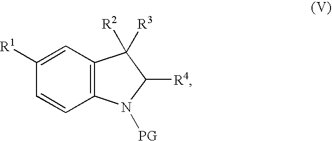
in which PG, R¹, R², R³ and R⁴ are each as defined in claim 1,
then removing the protective group PG using methods known from the literature, to give a compound of the formula (VI) 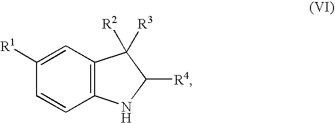
in which R¹, R², R³ and R⁴ are each as defined in claim 1,
then converting the compound, using a compound of the formula (VII) 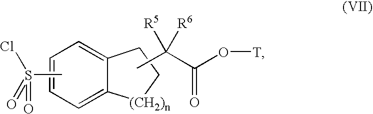
in which R⁵, R⁶ and n are each as defined in claim 1 and
T represents benzyl or (C₁-C₆)-alkyl,
in an inert solvent in the presence of a base into a compound of the formula (VIII) 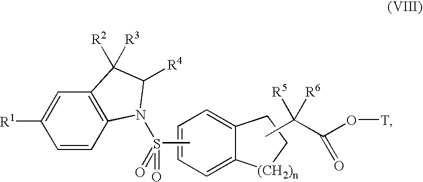
in which n, T, R¹, R², R³, R⁴, R⁵ and R⁶ are each as defined in claim 1,
then converting the compound of the formula (VIII) with acids or bases or, if T represents benzyl, also hydrogenolytically into the corresponding carboxylic acid of the formula (IX) 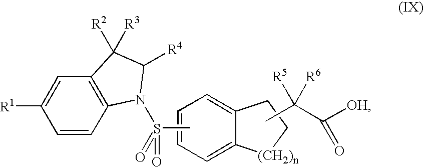
in which n, R¹, R², R³, R⁴, R⁵ and R⁶ are each as defined in claim 1,
further modifying this carboxylic acid (IX), if appropriate, using known esterification methods to give a compound of the formula (I),
and converting the resulting compound of the formula (IX) or (I), if appropriate, into a pharmaceutically acceptable salt thereof, using the corresponding bases or acids.

6. (canceled)

7. A pharmaceutical composition comprising a compound of claim 1 or 4 and inert non-toxic pharmaceutically acceptable carriers, auxiliaries, solvents, vehicles, emulsifiers and/or dispersants.

8. A method for treating or preventing stroke, arteriosclerosis, coronary heart disease or dyslipidaemia, comprising administering to a patient a therapeutically effective amount of a compound of claim 1 or 4.

9. (canceled)

10. (canceled)

11. (canceled)

12. A method for preventing myocardial infection, comprising administering to a patient a therapeutically effective amount of a compound of claim 1 or 4.

13. A method for treating restenosis after coronary angioplasty or stenting, comprising administering to a patient a therapeutically effective amount of a compound of claim 1 or 4.
